(19) United States
(12) Patent Application Publication
(10) Pub. No.: US 2026/0041800 A1
(43) Pub. Date: Feb. 12, 2026

(54) SOLID CHEMISTRY DISPENSING SYSTEM AND METHOD OF USE

(71) Applicant: ECOLAB USA INC., Saint Paul, MN (US)

(72) Inventors: Mark Newbold, BRISBANE (AU); Greg Dixon, HAMILTON (NZ)

(21) Appl. No.: 19/293,144

(22) Filed: Aug. 7, 2025

Related U.S. Application Data
(60) Provisional application No. 63/681,339, filed on Aug. 9, 2024.

(51) Int. Cl. A61L 2/18; A61L 2/26; B08B 3/08; B08B 13/00

(52) U.S. Cl. CPC A61L 2/18; A61L 2/26; B08B 3/08; B08B 13/00; A61L 2202/14; A61L 2202/17

(57) ABSTRACT

Disclosed herein are systems and equipment configured for dosing and dispensing solid cleaning compositions. The solid chemistry dispensing systems comprise a solids dispenser wherein a solid cleaning composition is stored and a tank connected to the solids dispenser. For dispensing and dosing, the tank is filled with a preselected volume of water. Said water is recirculated through the solids dispenser to dissolve the solid cleaning composition resulting in a concentrate of a cleaning composition in the tank.

### CROSS-REFERENCE TO RELATED APPLICATION

This application claims priority under 35 U.S.C. § 119 to Provisional Application U.S. Ser. No. 63/681,339, filed on Aug. 9, 2024, which is herein incorporated by reference in its entirety including without limitation, the specification, claims, and abstract, as well as any figures, tables, or examples thereof.

### TECHNICAL FIELD

The present disclosure relates generally to systems and equipment configured for dosing and dispensing solid cleaning compositions. The solid chemistry dispensing systems comprise a solids dispenser wherein a solid cleaning composition is stored and a tank connected to the solids dispenser. For dispensing and dosing, the tank is filled with a preselected volume of water. Said water is recirculated through the solids dispenser to dissolve the solid cleaning composition resulting in a concentrate of a cleaning composition in the tank. The concentrate can then be diluted into a use solution.

### BACKGROUND

The use of solid detergent and sanitizing products deliver desired cleaning and hygiene needs while also providing more sustainable products that significantly reduce plastic waste sources and disposal costs. Moreover, the use of solid compositions improves transport, overall application efficiency and worker safety, such as through reduced injuries from moving large liquid drums and/or splash hazards from the liquid compositions. For example, use of solid compositions reduces the required footprint for storage of liquid compositions, instead replacing with solids that can be diluted and used at a desired time.

CIP and other hard surface cleaning applications can benefit from solid cleaning compositions that replace liquid compositions. Food processing and other industries regularly use clean-in-place (CIP) clean-up procedures and automation. CIP systems are often found in industries which produce fluidized ingestible products for humans or animals such as the dairy industry, the pharmaceutical industry, and the food industry. CIP systems are generally regarded as large production plant systems having reservoirs, pipes, pumps and mixing vessels which cannot be broken down to be cleaned. In general, a typical CIP sequence may consist of the following five stages (see “Hygiene for Management” by R. A. Sprenger, 5th Edition, p. 135): 1. pre-rinse with cold water to remove gross soil; 2. detergent circulation to remove residual adhering debris and scale; 3. intermediate rinse with cold water to remove all traces of detergent; 4. disinfectant circulation to destroy remaining microorganisms; and 5. final rinse with cold water to remove all traces of disinfectants.

Unfortunately, reliably and repeatedly dissolving, dispensing, and dosing solid chemistries is challenging. There is a need for systems, equipment, and methods to accurately and reliably dispense and dose solid chemistries.

Therefore, it is an objective of the systems and methods described herein to provide a solid chemistry dispensing system and methods of use, for various applications, including CIP systems. Such systems and methods are water efficient and deliver a consistent quality cleaning and/or sanitizing regime.

Other objects, advantages and features of the present invention will become apparent from the following specification taken in conjunction with the accompanying drawings.

### SUMMARY

According to some embodiments of the present disclosure, the solid chemistry dispensing system comprises at least one solids dispenser configured for housing a solid chemistry, at least one concentrate tank, and a recirculation circuit configured to recirculate water between the concentrate tank and the solids dispenser. According to some additional embodiments of the present disclosure, the solid chemistry dispensing system comprises two or more solids dispenser each configured for housing a solid chemistry, two or more concentration tanks, and two or more recirculation circuits configured to recirculate water between each concentrate tank and solids dispenser.

According to some embodiments of the present disclosure, methods of dispensing a solid chemistry comprise placing a solid chemistry into the solids dispenser, adding a pre-determined volume of water to the concentrate tank, recirculating the water through the recirculation circuit, the solids dispenser, and the concentrate tank until the solid chemistry is the solids dispenser is substantially dissolved, and obtaining a concentrate in the concentrate tank.

According to some embodiments of the present disclosure, methods of cleaning and/or sanitizing a surface comprise contacting a surface in need of cleaning with the concentrate composition. In some embodiments, the concentrate composition is first diluted into a use solution before contacting a surface in need of cleaning and/or sanitizing.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

Several embodiments in which the present disclosure can be practiced are illustrated and described in detail, wherein like reference characters represent like components throughout the several views. The drawings are presented for exemplary purposes and may not be to scale unless otherwise indicated.

FIG. 1 is an exemplary solid chemistry dispensing system configured for dosing and dispensing two distinct solid chemistries.

An artisan of ordinary skill in the art need not view, within isolated figure(s), the near infinite distinct combinations of features described in the following detailed description to facilitate an understanding of the present disclosure.

### DETAILED DESCRIPTION

The embodiments are not limited to particular compositions and methods of use thereof, which can vary and are understood by skilled artisans. It is further to be understood that all terminology used herein is for the purpose of describing particular embodiments only, and is not intended to be limiting in any manner or scope. For example, as used in this specification and the appended claims, the singular forms “a,” “an” and “the” can include plural referents unless the content clearly indicates otherwise. Further, all units, prefixes, and symbols may be denoted in its SI accepted form.

Numeric ranges recited within the specification are inclusive of the numbers defining the range and include each integer within the defined range. Throughout this disclosure, various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges, fractions, and individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6, and decimals and fractions, for example, 1.2, 3.8, 11/2, and 43/4. This applies regardless of the breadth of the range.

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

The term “about,” as used herein, refers to variation in the numerical quantity that can occur, for example, through typical measuring techniques and equipment, with respect to any quantifiable variable, including, but not limited to, concentration, mass, volume, time, temperature, pH, log count of bacteria or viruses, and the like. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations. Whether or not modified by the term “about,” the claims include equivalents to the quantities.

Differentiation of antimicrobial “-cidal” or “-static” activity, the definitions which describe the degree of efficacy, and the official laboratory protocols for measuring this efficacy are considerations for understanding the relevance of antimicrobial agents and compositions. Antimicrobial compositions can affect two kinds of microbial cell damage. The first is a lethal, irreversible action resulting in complete microbial cell destruction or incapacitation. The second type of cell damage is reversible, such that if the organism is rendered free of the agent, it can again multiply. The former is termed microbiocidal and the later, microbistatic. A sanitizer and a disinfectant are, by definition, agents which provide antimicrobial or microbiocidal activity. As used herein, the term “sanitizer” refers to an agent that reduces the number of bacterial contaminants to safe levels as judged by public health requirements. These reductions can be evaluated using a procedure set out in Germicidal and Detergent Sanitizing Action of Disinfectants, Official Methods of Analysis of the Association of Official Analytical Chemists, paragraph 960.09 and applicable sections, 15th Edition, 1990 (EPA Guideline 91-2). According to this reference a sanitizer should provide a 99.999% reduction (5-log order reduction) within 30 seconds at room temperature, 25±2° C., against several test organisms, including Pseudomonas, E. coli, and Staph.

As used herein, the term “sanitizing” refers to a method used to facilitate or aid in antimicrobial activity.

As used herein, the term “cleaning” refers to a method used to facilitate or aid in soil removal, bleaching, microbial population reduction, rinsing, and any combination thereof.

As used herein, the term “disinfectant” refers to an agent that kills all vegetative cells including most recognized pathogenic microorganisms. In an embodiment, a disinfectant according to U.S. standards can use the procedure described in A.O.A.C. Use Dilution Methods, Official Methods of Analysis of the Association of Official Analytical Chemists, paragraph 955.14 and applicable sections, 15th Edition, 1990 (EPA Guideline 91-2). As used herein, the term “high level disinfection” or “high level disinfectant” refers to a compound or composition that kills substantially all organisms, except high levels of bacterial spores, and is affected with a chemical germicide cleared for marketing as a sterilant by the Food and Drug Administration. As used herein, the term “intermediate-level disinfection” or “intermediate level disinfectant” refers to a compound or composition that kills mycobacteria, most viruses, and bacteria with a chemical germicide registered as a tuberculocide by the Environmental Protection Agency (EPA). As used herein, the term “low-level disinfection” or “low level disinfectant” refers to a compound or composition that kills some viruses and bacteria with a chemical germicide registered as a hospital disinfectant by the EPA.

As used herein, the term “exemplary” refers to an example, an instance, or an illustration, and does not indicate a most preferred embodiment unless otherwise stated.

As used herein, the phrase “food processing surface” refers to a surface of a tool, a machine, equipment, a structure, a building, or the like that is employed as part of a food processing, preparation, or storage activity. Examples of food processing surfaces include surfaces of food processing or preparation equipment (e.g., slicing, canning, or transport equipment, including flumes), of food processing wares (e.g., utensils, dishware, wash ware, and bar glasses), and of floors, walls, or fixtures of structures in which food processing occurs. Food processing surfaces are found and employed in food anti-spoilage air circulation systems, aseptic packaging sanitizing, food refrigeration and cooler cleaners and sanitizers, ware washing sanitizing, blancher cleaning and sanitizing, food packaging materials, cutting board additives, third-sink sanitizing, beverage chillers and warmers, meat chilling or scalding waters, autodish sanitizers, sanitizing gels, cooling towers, food processing antimicrobial garment sprays, and non-to-low-aqueous food preparation lubricants, oils, and rinse additives.

The term “hard surface” refers to a solid, substantially non-flexible surface such as a countertop, tile, floor, wall, panel, window, plumbing fixture, kitchen and bathroom furniture, appliance, engine, circuit board, and dish. Hard surfaces may include for example, health care surfaces and food processing surfaces.

As used herein, the phrase “health care surface” refers to a surface of an instrument, a device, a cart, a cage, furniture, a structure, a building, or the like that is employed as part of a health care activity. Examples of health care surfaces include surfaces of medical or dental instruments, of medical or dental devices, of electronic apparatus employed for monitoring patient health, and of floors, walls, or fixtures of structures in which health care occurs. Health care surfaces are found in hospital, surgical, infirmity, birthing, mortuary, and clinical diagnosis rooms. These surfaces can be those typified as “hard surfaces” (such as walls, floors, bed-pans, etc.,), or fabric surfaces, e.g., knit, woven, and non-woven surfaces (such as surgical garments, draperies, bed linens, bandages, etc.,), or patient-care equipment (such as respirators, diagnostic equipment, shunts, body scopes, wheelchairs, beds, etc.,), or surgical and diagnostic equipment. Health care surfaces include articles and surfaces employed in animal health care.

The term “generally” encompasses both “about” and “substantially.”

The terms “include” and “including” when used in reference to a list of materials refer to but are not limited to the materials so listed.

The term “or” is synonymous with “and/or” and means any one member or combination of members of a particular list.

The “scope” of the present disclosure is defined by the appended claims, along with the full scope of equivalents to which such claims are entitled. The scope of the disclosure is further qualified as including any possible modification to any of the aspects and/or embodiments disclosed herein which would result in other embodiments, combinations, subcombinations, or the like that would be obvious to those skilled in the art.

As used herein, the term “soil” refers to polar or non-polar organic or inorganic substances including, but not limited to carbohydrates, proteins, fats, oils and the like. These substances may be present in their organic state or complexed to a metal to form an inorganic complex.

The term “substantially” refers to a great or significant extent. “Substantially” can thus refer to a plurality, majority, and/or a supermajority of said quantifiable variables, given proper context.

As used herein, the term “substantially free” refers to compositions completely lacking the component or having such a small amount of the component that the component does not affect the performance of the composition. The component may be present as an impurity or as a contaminant and shall be less than 0.5 wt-%. In another embodiment, the amount of the component is less than 0.1 wt-% and in yet another embodiment, the amount of component is less than 0.01 wt-%.

The term “weight percent,” “wt-%,” “percent by weight,” “% by weight,” and variations thereof, as used herein, refer to the concentration of a substance as the weight of that substance divided by the total weight of the composition and multiplied by 100. It is understood that, as used here, “percent,” “%,” and the like are intended to be synonymous with “weight percent,” “wt-%,” etc.

The methods and compositions of the present invention may comprise, consist essentially of, or consist of the components and ingredients of the present invention as well as other ingredients described herein. As used herein, “consisting essentially of” means that the methods and compositions may include additional steps, components or ingredients, but only if the additional steps, components or ingredients do not materially alter the basic and novel characteristics of the claimed methods and compositions.

It should also be noted that, as used in this specification and the appended claims, the term “configured” describes a system, apparatus, or other structure that is constructed or configured to perform a particular task or adopt a particular configuration. The term “configured” can be used interchangeably with other similar phrases such as arranged and configured, constructed and arranged, adapted and configured, adapted, constructed, manufactured and arranged, and the like.

### Solid Chemistry Dispensing System

Disclosed herein are systems configured for dispensing and dosing solid chemistries. Solid chemistry dispensing systems comprise a solids dispenser configured to house a solid chemistry, a concentrate tank, and a recirculation circuit configured to recirculate water between the concentrate tank and the solids dispenser. In some embodiments, the solid chemistry dispensing system comprises at least one solids dispenser configured for housing a solid chemistry, at least one concentrate tank, and at least one recirculation circuit configured to recirculate water between the concentrate tank and the solids dispenser. In some embodiments, the solid chemistry dispensing system comprises more than one solids dispenser each configured for housing a solid chemistry, more than one concentrate tank, and more than one recirculation circuit configured to recirculate water between the concentrate tank and the solids dispenser. In such embodiments, each solids dispenser houses a distinct solid chemistry, and each solids dispenser is connected to one concentrate tank and one recirculation circuit. The solid chemistry dispensing system as described herein can comprise as many solids dispensers, concentration tanks, and recirculation circuits as needed. For example, the system can be configured to dispense one, two, three, four, five, six, up to ten, or more than 10 solid chemistries.

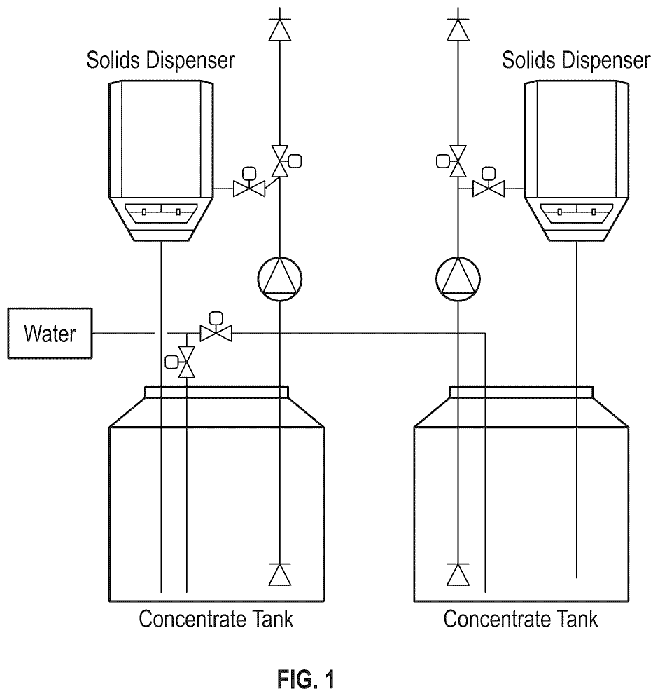

An exemplary solid chemistry dispensing system is shown in FIG. 1. FIG. 1 shows a schematic of a system configured to dose two distinct solid chemistries. The system is configured to dispense water into the concentrate tank. The system is configured to recirculate water from the concentrate tank, through the solids dispenser, and back into the concentrate tank. The system is configured to recirculate the water through the solids dispenser and concentrate tank long enough to ensure that the solid chemistry is substantially dissolved into the water. The recirculation circuit is configured such that after the solid chemistry is substantially dissolved, recirculation ceases resulting in a concentrate composition contained within the concentrate tank. The concentrate composition can then be used for cleaning and/or sanitizing, or further diluted into a use solution and then used for cleaning and/or sanitizing.

The solids dispenser as described herein can comprise any shape or volume or material as known in the art and that is compatible with the solid chemistry stored therein. In some embodiments, the solids dispenser comprises a plastic material. In some embodiments, the solids dispenser comprises a lid, wherein a user opens a lid to place a solid chemistry therein and wherein the lid is configured such that when closed the solid dispenser is substantially or entirely enclosed.

The concentrate tank as described herein can comprise any shape or volume or material as known in the art and that is compatible with the solid chemistry dissolved into a concentrate. In some embodiments, the concentrate tank comprises a plastic material. In some embodiments the concentrate tank has a volume capacity of from about 1 liter to about 1000 liters. In some embodiments, the concentrate tank is substantially or entirely enclosed.

The recirculation circuit as disclosed herein can comprise any shape or volume or material as known in the art and that is compatible with the solid chemistry dissolved into a concentrate. The recirculation circuit as disclosed herein can comprise any type of vehicle for fluid flow, controlling said fluid flow, and ceasing said fluid flow. In some embodiments the recirculation circuit comprises any type of pipes, hoses, valves, sprays and/or pumps, and the like as known in the art. In some embodiments, the recirculation circuit comprises plastic pipes. The recirculation circuit is configured to recirculate water through the solids dispenser and the concentrate tank and is configured such that after the solid chemistry is substantially dissolved, recirculation ceases resulting in a concentrate composition contained within the concentrate tank

In some embodiments, the solid chemistry dispensing system further comprises a water intake configured to deliver a volume of water to the concentrate tank. The water intake can comprise any shape or material as known in the art, for example a pipe or hose and a pump, valves, and the like. In embodiments dosing more than one solid chemistry, each concentrate tank can have the same water intake as any other concentrate tank, or a distinct water intake.

In some embodiments, the dispensing system comprises a controller unit. A controller unit may comprise an interface wherein water fill time, recirculation time, and dosing period can be independently configured. In some embodiments, the controller unit comprises the main power supply. In some embodiments, the controller unit comprises switches for each process step. In some embodiments, the controller unit comprises emergency protection, for example a fast-stop off switch. In some embodiments, the controller unit is configured for a user to select a pre-determined volume of water to obtain a selected concentrate of solid chemistry in the concentrate composition in the concentrate tank when the solid chemistry is substantially dissolved. In some embodiments, the controller unit is configured to control any of the recirculation units to recirculate the water for a pre-determined amount of time, sufficient for the solid chemistry to be substantially dissolved.

The dispensing system as described herein may comprise any number of safety or monitoring features, including but not limited to monitoring temperature, pressure, volume, and the like. Such features can include stopping the dispensing system when a monitored parameter is considered outside desired operating conditions. Such features can also include correcting a parameter considered outside desired operating conditions.

### Method of Use

Disclosed herein are methods of dispensing a solid chemistry utilizing the solid chemistry dispensing system as described herein. The method comprises placing a solid chemistry into the solids dispenser and then adding a pre-selected volume of water to the concentrate tank. The volume of water is selected to obtain a desired concentration of chemistry in said water; the solid chemistry is substantially dissolved in said volume of water to obtain a desired concentration of chemistry in an aqueous solution. The method comprises circulating the water through the recirculation circuit, the solid dispenser, and the concentrate tank until the solid chemistry within the solid dispenser is substantially dissolved in the water. Substantial dissolution as used herein means the solid chemistry is at least about 95 wt-%, at least 96 wt-%, at least about 97 wt-%, at least about 98 wt-%, or at least about 99 wt-% dissolved into the water. In some embodiments the solid chemistry is entirely, or about 100 wt-%, dissolved into the water.

In some embodiments, the method comprises recirculating the water through the recirculation circuit for a pre-determined amount of time. In these embodiments, the pre-determined amount of time is dependent upon the dissolution rate of the solid chemistry. The pre-determined amount of time is selected to ensure substantial dissolution of the solid chemistry into solution to achieve the required concentration of chemistry in the concentrate solution. In some embodiments, the amount of time the water is recirculated is about 1 minute to about 120 minutes, or about 10 minutes to about 100 minutes, or from about 60 minutes to about 90 minutes.

The recirculation circuit can introduce the water into the solid dispenser by any method as known in the art to dissolve a solid chemistry. In some embodiments, the recirculation circuit sprays the water onto the solid chemistry. In some embodiments, the recirculation circuit passes the water over a portion of the solid chemistry. In some embodiments, the recirculation circuit submerges the solid chemistry in water. In some embodiments, the solids dispenser and/or the recirculation circuit comprise agitation to accelerate dissolution of the solid chemistry.

In some embodiments, the method comprises ceasing recirculation after substantially dissolving the solid chemistry into the water, and obtaining a concentrate composition in the concentrate tank. The concentrate composition can then be utilized for cleaning and/or sanitizing.

In methods wherein the dispensing system is configured to dispense more than one distinct solid chemistry, the chemistries can be dispensed concurrently or independently. In some embodiments, the systems and methods are configured to dispense one solid chemistry at a time. In some embodiments, two or more solid chemistry systems are utilized in the same facility. For example, a facility may utilize a solid alkali chemistry such as a concentrated caustic-based cleaning product designed for use against fat and protein soils, and a solid acid chemistry as a detergent sanitizer to inhibit and control the growth of microorganisms. As another example, a facility may utilize a solid alkali chemistry, a solid acid chemistry, and a solid surfactant package to further aid with soil removal. In such embodiments, the solid chemistry dispensing system may dispense and dose the solid chemistries at the same time, or at separate times.

In some embodiments, the method comprises diluting the concentrate composition into a use solution. The water that is used to dilute the concentrate to form the use composition can be referred to as water of dilution or a diluent. The typical dilution factor is between approximately 1 and approximately 10,000. In an embodiment, the concentrate is diluted into a liquid use solution at a ratio of between about 1:10 and about 1:10,000 concentrate to water, between about 1:10 and about 1:1,000 concentrate to water, or between about 1:10 and about 1:500 concentrate to water. The concentrate compositions can be diluted using any type of manual or automated dispensing system and stored prior to use. In some embodiments the concentrate composition is diluted with water and stored in a tank, such as a day tank, having any desired volume, e.g. 500 L or more, 500-1000 L or more, etc.

Water temperature conditions for the methods described herein can range from about 4° C. to about 90° C., about 10° C. to about 90° C., or about 20° C. to about 80° C.

The methods can optionally include the use of various sensors and/or indicators. In an embodiment, the level of active ingredients in the concentrate composition or use solution can be monitored by various ways as known in the art. In some embodiments, properties of the concentrate composition or use solution including pH, anionic activity, fluorescence, and/or conductivity can be monitored by sensors that provide a visual or audible signal when the composition is not within a specified range. In some embodiments, a marker molecule can be added to the composition, where the change of the active ingredients in the concentrate or use solution will trigger the physical and/or chemical property changes of the marker molecule, and the change is quantified through a signal processing.

In some embodiments, the method comprises a pre-check prior to adding the pre-selected volume of water to the concentrate take. In some embodiments, the pre-check comprises checking the level of concentrate and/or water in a concentrate tank to ensure capacity for said pre-selected volume of water. In these embodiments, if sufficient capacity cannot be verified, the method does not commence. In some embodiments, the pre-check comprises ensuring that the solid dispenser comprises the solid chemistry.

### Method of Cleaning

Described herein is a method of cleaning and/or sanitizing a surface comprising contacting the surface with the concentrate composition and/or use solution as described herein. The concentrate compositions or use solutions described herein are particularly beneficial as cleaning compositions for various clean-in-place “CIP” systems. CIP systems typically include a pre-rinse to remove soils, detergent circulation to remove residual adhering debris and scale, an intermediate rinse to remove the detergent, a disinfectant circulation, and a final rinse. The concentrate composition or use solution is dosed through a CIP system using pump recirculation systems during the cleaning program for soil removal and/or sanitizing.

In preferred embodiments, the method comprises a solid alkali chemistry such as a concentrated caustic-based cleaning product for cleaning fat and protein soils, and a solid acid chemistry as a detergent sanitizer to inhibit and control the growth of microorganisms. Such embodiments are particularly beneficial in CIP processes in the dairy industry and other food processing facilities.

In an embodiment, the concentrate compositions or use solutions are also useful in cleaning various hard surfaces in institutional and non-institutional environments.

In embodiments contacting the concentrate composition or use solution of the solid composition can be for treating surfaces in need of antimicrobial efficacy, including for example virucidal efficacy. In further aspects, contacting the concentrate composition or use solution can be for treating surfaces in need of virucidal efficacy against small, non-enveloped viruses, large, non-enveloped viruses and/or any enveloped viruses. In a particular aspect, contacting the use solution of the solid composition can be for treating surfaces in need of inactivating small, non-enveloped viruses including Norovirus.

In a further aspect, contacting the concentrate composition or use solution can be to a surface contaminated with a biofilm. As referred to herein, a biofilm is often formed on surfaces in contact with water, providing a hydrated matrix of polysaccharides to provide structural protection from biocides and antimicrobial agents, making biofilm more difficult to kill than other pathogens. The contacting step can include providing the concentrate composition or use solution to a hard surface in contact with biofilm, such as for example, walls, floors, sinks, countertops, drain lines, pipes and other plumbing surfaces, tubes and valves and like. Exemplary industries in which the methods and compositions may be used include, but are not limited to, institutional industries, including hotels, housekeeping and foodservice, food processing, water care industries, janitorial industries, and health care. According to embodiments of the methods, the contacting step reduces and/or eliminating biofilm growth produced by a wide variety of bacteria and other microorganisms.

In a further aspect, contacting the concentrate composition or use solution can be to a food contact and/or non-food contact hard surface. Such surfaces can further include instruments, such as medical instruments. Surfaces can also include those cleaned in third-sink sanitizing, warewashing applications, including various wares. Surfaces can also include dish machines, such as for deliming and/or cleaning the inside surfaces thereof. Still further surfaces can include floors and other hard surfaces.

The various surfaces to which the compositions can be applied can include any conventional application means. In CIP systems, the concentrate composition or use solution is pumped or otherwise distributed through said system. In these embodiments, the concentrate composition or use solutions contacts the system for at least about 1 minute, or from about 1 minute to about 1 hour, or any amount sufficient for removing soil and/or sanitizing. In non-CIP systems applications can include, for example, wiping, spraying, dipping, immersing, or the like with the concentrate composition or use solution of the solid composition or soaking with the use solution. The contacting step allows the composition to contact the soiled surface for a predetermined amount of time. The amount of time can be sufficient to allow soil removal or sanitization, including from a few seconds to several hours, from about 15 seconds, or about 30 seconds to about 3 hours, or any range there-between. In a preferred embodiment, the contact time required for antiviral efficacy is less than about 30 minutes. The methods may comprise a single step of applying the composition onto the surface without direct physical removal, such as a rinse step and/or a wiping step.

### Solid Compositions

Any solid detergent, cleaning or sanitizing chemistry that dissolves in water to form a concentrate composition or use solution may be utilized in the systems or methods described herein. The solid chemistries may comprise various functional ingredients including for example, detergent or sanitizing components. For the purpose of this application, the term “functional ingredient” includes a material that when dispersed or dissolved in a use and/or concentrate solution, such as an aqueous solution, provides a beneficial property in a particular use. Some particular examples of functional materials include, for example, an acid source, an alkaline source, defoaming agents (including defoaming surfactants), surfactants (including detersive surfactants), wetting agents, anti-redeposition agents, solubility modifiers, inorganic fillers, dispersants, rinse aids, metal protecting agents, stabilizing agents, corrosion inhibitors, sequestrants and/or chelating agents, threshold agent, fragrances and/or dyes, rheology modifiers or thickeners, hydrotropes or couplers, buffers, solvents (including water), solidification agents, and the like.

For the purpose of the present description solid compositions refer to those that are “solid” at use temperature, is sufficiently resistant to flow such that the unsupported composition will not substantially change shape upon standing. Such a solid can be for example, in the form of a hard block or brick, a cast solid, or pressed solid. As one skilled in the art will ascertain the size and shape of the solid composition can vary based on an application of use.

In some embodiments the solid compositions are cast solids based on the wt-% of liquid components contained therein. Solids can be made in varying sizes and shapes. In some embodiments the solid are blocks from about 1 kg to about 10 kg, or about 2 kg to about 5 kg. As one skilled in the art will ascertain the size and shape of the solid composition can vary based on an application of use.

In some embodiments the hardness of the solid compositions has a penetrometer measurement of about 400 mm or less. In other embodiments the hardness of the solid composition has a penetrometer measurement of about 200 mm or less.

All publications and patent applications in this specification are indicative of the level of ordinary skill in the art to which this invention pertains. All publications and patent applications are herein incorporated by reference to the same extent as if each individual publication or patent application were specifically and individually indicated as incorporated by reference.

### Embodiments

The present disclosure is further defined by the following numbered embodiments:

1. A solid chemistry dispensing system comprising: at least one solids dispenser configured for housing a solid chemistry; at least one concentrate tank; at least one recirculation circuit configured to recirculate water between the concentrate tank and the solids dispenser.

2. A solid chemistry dispensing system comprising: a first solids dispenser configured for housing a first solid chemistry; a first concentrate tank; a first recirculation circuit configured to recirculate water between the first concentrate tank and the first solids dispenser.

3. The solid chemistry dispensing system of embodiment 2, further comprising: a second solids dispenser configured for housing a second solid chemistry; a second concentrate tank; a second recirculation circuit configured to recirculate water between the second concentrate tank and the second solids dispenser.

4. The solid chemistry dispensing system of embodiment 3, wherein the first solid chemistry is an acid solid chemistry, and the second solid chemistry is an alkaline solid chemistry.

5. The solid chemistry dispensing system of any one of embodiments 2-4, further comprising: a third solids dispenser configured for housing a third solid chemistry; a third concentrate tank; a third recirculation circuit configured to recirculate water between the third concentrate tank and the third solids dispenser.

6. The solid chemistry dispensing system of any one of embodiments 1-5, further comprising a water intake configured for delivering a volume of water into each concentrate tank.

7. The solid chemistry dispensing system of embodiment 6, wherein each concentrate tank has a distinct water intake.

8. The solid chemistry dispensing system of embodiment 6, wherein the concentrate tanks have the same water intake.

9. The solid chemistry dispensing system of any one of embodiments 1-8, further comprising a controller unit.

10. The solid chemistry dispensing system of embodiment 9, wherein the controller unit is configured for a user to select a pre-determined volume of water for a pre-determined concentration of solid chemistry in a concentrate composition in the concentrate tank.

11. The solid chemistry dispensing system of any one of embodiments 9-10, wherein the controller unit is configured to control any of the recirculation units to recirculate the water for a pre-determined amount of time.

12. The solid chemistry dispensing system of embodiment 11, wherein the pre-determined amount of time is selected to ensure that at least about 99 wt-% of the solid chemistry is dissolved into the water.

13. A method of dispensing a solid chemistry comprising: obtaining the solid chemistry dispensing system of any one of embodiments 1-12; placing a solid chemistry into the solids dispenser; adding a pre-determined volume of water to the concentrate tank, wherein the pre-determined volume of water is selected to ensure a pre-determined concentration of chemistry in the water; recirculating the volume of water through the recirculation circuit, the solids dispenser and the concentrate tank until the solid chemistry in the solids dispenser is substantially dissolved; obtaining a concentrate composition in the concentrate tank.

14. The method of embodiment 13, wherein the volume of water is recirculated for a pre-determined amount of time.

15. The method of embodiment 14, wherein the pre-determined amount of time is selected to ensure the solid chemistry is substantially dissolved.

16. The method of any one of embodiments 13-15, wherein the solid chemistry dispensing system comprises a first and a second solid chemistry dispensing system, wherein the first and second solid chemistries are distinct, and wherein the two are not dispensed at the same time.

17. The method of any one of embodiments 13-16, wherein the solid chemistry dispensing system comprises a first, a second, and a third solid chemistry dispensing system, wherein the first, second and third chemistries are distinct, and wherein none of the three are dispensed at the same time.

18. The method of any one of embodiments 13-17, wherein the solid chemistry is at least about 99 wt-% dissolved in the pre-determined volume of water.

19. The method of any one of embodiments 13-18, wherein the solid chemistry is at least about 100 wt-% dissolved in the predetermined volume of water.

20. A method of cleaning and/or sanitizing a surface comprising: obtaining the concentrate composition from the method of any one of embodiments 13-19; and contacting a surface in need of cleaning and/or sanitizing.

21. The method of embodiment 20, further comprising diluting the concentrate composition into a use solution before contacting a surface in need of cleaning.

22. The method of embodiment 21, wherein the concentrate composition is diluted into the use solution at a ratio of between about 1:10 and about 1:10,000 concentrate to water, between about 1:10 and about 1:1,000 concentrate to water, or between about 1:10 and about 1:500 concentrate to water.

23. The method of any one of embodiments 20-22, wherein the surface is a hard surface.

24. The method of any one of embodiments 20-23, wherein the surface in need of cleaning comprises a food processing surface.

25. The method of any one of embodiments 20-24, wherein the surface is a clean-in-place system.

26. The method of any one of embodiments 20-25, wherein the surface is part of a dairy operation or facility.

### EXAMPLES

Embodiments of the present invention are further defined in the following non-limiting Examples. It should be understood that these Examples, while indicating certain embodiments of the invention, are given by way of illustration only. From the above discussion and these Examples, one skilled in the art can ascertain the essential characteristics of this invention, and without departing from the spirit and scope thereof, can make various changes and modifications of the embodiments of the invention to adapt it to various usages and conditions. Thus, various modifications of the embodiments, in addition to those shown and described herein, will be apparent to those skilled in the art from the foregoing description. Such modifications are also intended to fall within the scope of the appended claims.

### Example 1—Dispensing an Acid Solid Chemistry

The dispensing units were tested at various locations to confirm successful dispensing and dosing of solid acid chemistries. At each site, a capsule comprising a solid acid chemistry was placed into the solids dispenser. Water sufficient to achieve a 13.5 to 15% w/v concentrate was added to the 20 liter concentrate tank. The water was recirculated through the solids dispenser and the concentrate tank for about 60 to 90 minutes until the capsule was fully dissolved into the water. The resultant concentrate composition in the concentrate tank was recorded. For use, a portion of the concentrate was diluted with 140 to 600 liters of 80° C. water to achieve use solutions with a concentration of 0.05 to 0.15% w/v. The use solutions were used as a part of the CIP processes at several dairy milking facilities, labeled A through J. The parameters for each test are shown in Table 1. The dispensing and dosing units reliably dispensed solid acid chemistries into a concentrate and reliably dosed said concentrate into a use solution.

TABLE 1

| Dairy | | | | | Quantity |
|---|---|---|---|---|---|
| Facility | | | | | of |
| and | | | | | Concentrate |
| Test | Capsule | Concentrate | Dilution | Use Solution | in Use |
| Number | Size | Composition | Volume | Concentration | Solution |
| A | 2.7 kg | 13.5% w/v | 600 L | 0.1% w/v | 4.4 L |
| B - 1 | 2.7 kg | 13.5% w/v | 450 L | 0.1% w/v | 3.3 L |
| B - 2 | 2.7 kg | 13.5% w/v | 325 L | 0.1% w/v | 2.4 L |
| B - 3 | 2.7 kg | 13.5% w/v | 140 L | 0.1% w/v | 1.0 L |
| B - 4 | 3 kg | 15% w/v | 450 L | 0.05% w/v | 1.5 L |
| B - 5 | 3 kg | 15% w/v | 325 L | 0.05% w/v | 1.1 L |
| B - 6 | 3 kg | 15% w/v | 140 L | 0.05% w/v | 0.5 L |
| C - 1 | 2.7 kg | 13.5% w/v | 600 L | 0.15% w/v | 6.7 L |
| C - 2 | 2.7 kg | 13.5% w/v | 400 L | 0.15% w/v | 4.4 L |
| D - 1 | 2.7 kg | 13.5% w/v | 540 L | 0.1% w/v | 4 L |
| D - 2 | 2.7 kg | 13.5% w/v | 430 L | 0.1% w/v | 3.2 L |
| E - 1 | 2.7 kg | 13.5% w/v | 450 L | 0.1% w/v | 3.3 L |
| E - 2 | 2.7 kg | 13.5% w/v | 420 L | 0.1% w/v | 3.1 L |
| F - 1 | 2.7 kg | 13.5% w/v | 450 L | 0.1% w/v | 3.3 L |
| F - 2 | 2.7 kg | 13.5% w/v | 580 L | 0.1% w/v | 4.3 L |
| G - 1 | 2.7 kg | 13.5% w/v | 600 L | 0.1% w/v | 4.4 L |
| G - 2 | 2.7 kg | 13.5% w/v | 250 L | 0.1% w/v | 1.9 L |
| H - 1 | 2.7 kg | 13.5% w/v | 250 L | 0.1% w/v | 1.9 L |
| H - 2 | 2.7 kg | 13.5% w/v | 250 L | 0.1% w/v | 1.9 L |
| I - 1 | 2.7 kg | 13.5% w/v | 500 L | 0.1% w/v | 3.7 L |
| I - 2 | 2.7 kg | 13.5% w/v | 250 L | 0.1% w/v | 1.9 L |
| J - 1 | 2.7 kg | 13.5% w/v | 450 L | 0.1% w/v | 3.3 L |
| J - 2 | 2.7 kg | 13.5% w/v | 325 L | 0.1% w/v | 2.4 L |

### Example 2—Dispensing an Alkaline Solid Chemistry

The dispensing units were tested at various locations to confirm successful dispensing and dosing of solid alkaline chemistries. At each site, a capsule comprising a solid alkaline chemistry was placed into the solids dispenser. Water sufficient to achieve a 20 to 21% w/v concentrate was added to the 20 liter concentrate tank. The water was recirculated through the solids dispenser and the concentrate tank for about 60 to 90 minutes until the capsule was fully dissolved into the water. The resultant concentrate composition in the concentrate tank was recorded. For use, a portion of the concentrate was diluted with 140 to 600 liters of 80° C. water to achieve use solutions with a concentration of 0.1 to 0.2% w/v. The use solutions were used as a part of the CIP processes at several dairy milking facilities. The parameters for each test are shown in Table 2. The dispensing and dosing units reliably dispensed solid alkaline chemistries into a concentrate and reliably dosed said concentrate into a use solution.

TABLE 2

| Dairy | | | | | Quantity |
|---|---|---|---|---|---|
| Facility | | | | | of |
| and | | | | | Concentrate |
| Test | Capsule | Concentrate | Dilution | Use Solution | in Use |
| Number | Size | Composition | Volume | Concentration | Solution |
| A | 4.1 kg | 21% w/v | 600 L | 0.15% w/v | 4.4 L |
| B - 1 | 4.1 kg | 21% w/v | 450 L | 0.15% w/v | 3.3 L |
| B - 2 | 4.1 kg | 21% w/v | 325 L | 0.15% w/v | 2.4 L |
| B - 3 | 4.1 kg | 21% w/v | 140 L | 0.15% w/v | 1.0 L |
| B - 4 | 4 kg | 20% w/v | 450 L | 0.1% w/v | 2.3 L |
| B - 5 | 4 kg | 20% w/v | 325 L | 0.1% w/v | 1.6 L |
| B - 6 | 4 kg | 20% w/v | 140 L | 0.1% w/v | 0.7 L |
| C - 1 | 4.1 kg | 21% w/v | 600 L | 0.15% w/v | 4.4 L |
| C - 2 | 4.1 kg | 21% w/v | 400 L | 0.15% w/v | 2.9 L |
| D - 1 | 4.1 kg | 21% w/v | 540 L | 0.15% w/v | 4.0 L |
| D - 2 | 4.1 kg | 21% w/v | 430 L | 0.15% w/v | 3.1 L |
| E - 1 | 4.1 kg | 21% w/v | 450 L | 0.15% w/v | 3.3 L |
| E - 2 | 4.1 kg | 21% w/v | 420 L | 0.15% w/v | 3.1 L |
| F - 1 | 4.1 kg | 21% w/v | 450 L | 0.15% w/v | 3.3 L |
| F - 2 | 4.1 kg | 21% w/v | 580 L | 0.15% w/v | 4.2 L |
| G - 1 | 4.1 kg | 21% w/v | 600 L | 0.15% w/v | 4.4 L |
| G - 2 | 4.1 kg | 21% w/v | 250 L | 0.15% w/v | 1.8 L |
| H | 4.1 kg | 21% w/v | 250 L | 0.2% w/v | 2.4 L |
| I - 1 | 4.1 kg | 21% w/v | 500 L | 0.2% w/v | 4.9 L |
| I - 2 | 4.1 kg | 21% w/v | 250 L | 0.2% w/v | 2.4 L |
| J | 4.1 kg | 21% w/v | 250 L | 0.2% w/v | 2.4 L |

The inventions being thus described, it will be obvious that the same may be varied in many ways. Such variations are not to be regarded as a departure from the spirit and scope of the inventions and all such modifications are intended to be included within the scope of the following claims. The above specification provides a description of the manufacture and use of the disclosed compositions and methods. Since many embodiments can be made without departing from the spirit and scope of the invention, the invention resides in the claims.

What is claimed is:

1. A solid chemistry dispensing system comprising:
a first solids dispenser configured for housing a first solid chemistry;
a first concentrate tank;
a first recirculation circuit configured to recirculate water between the first concentrate tank and the first solids dispenser.

2. The solid chemistry dispensing system of claim 1, further comprising:
a second solids dispenser configured for housing a second solid chemistry;
a second concentrate tank;
a second recirculation circuit configured to recirculate water between the second concentrate tank and the second solids dispenser.

3. The solid chemistry dispensing system of claim 2, wherein the first solid chemistry is an acid solid chemistry, and the second solid chemistry is an alkaline solid chemistry.

4. The solid chemistry dispensing system of claim 2, further comprising:
a third solids dispenser configured for housing a third solid chemistry;
a third concentrate tank;
a third recirculation circuit configured to recirculate water between the third concentrate tank and the third solids dispenser.

5. The solid chemistry dispensing system of any claim 2, further comprising a water intake configured for delivering a volume of water into each concentrate tank.

6. The solid chemistry dispensing system of claim 5, wherein each concentrate tank has a distinct water intake, or wherein the concentrate tanks heave the same water intake.

7. The solid chemistry dispensing system of claim 1, further comprising a controller unit.

8. The solid chemistry dispensing system of claim 7, wherein the controller unit is configured for a user to select a pre-determined volume of water for a pre-determined concentration of solid chemistry in a concentrate composition in the concentrate tank.

9. The solid chemistry dispensing system of claim 7, wherein the controller unit is configured to control any of the recirculation units to recirculate the water for a pre-determined amount of time.

10. The solid chemistry dispensing system of claim 9, wherein the pre-determined amount of time is selected to ensure that at least about 99 wt-% of the solid chemistry is dissolved into the water.

11. A method of dispensing a solid chemistry comprising:
obtaining a solid chemistry dispensing system comprising:
at least one solids dispenser configured for housing a solid chemistry;
at least one concentrate tank;
at least one recirculation circuit configured to recirculate water between the concentrate tank and the solids dispenser;
placing a solid chemistry into the solids dispenser;
adding a pre-determined volume of water to the concentrate tank, wherein the pre-determined volume of water is selected to ensure a pre-determined concentration of chemistry in the water;
recirculating the volume of water through the recirculation circuit, the solids dispenser and the concentrate tank until the solid chemistry in the solids dispenser is substantially dissolved;
obtaining a concentrate composition in the concentrate tank.

12. The method of claim 11, wherein the volume of water is recirculated for a pre-determined amount of time, wherein the pre-determined amount of time is selected to ensure the solid chemistry is substantially dissolved.

13. The method of claim 11, wherein the solid chemistry dispensing system comprises a first and a second solid chemistry dispensing system, wherein the first and second solid chemistries are distinct, and wherein the two are not dispensed at the same time.

14. The method of claim 11, wherein the solid chemistry dispensing system comprises a first, a second, and a third solid chemistry dispensing system, wherein the first, second and third chemistries are distinct, and wherein none of the three are dispensed at the same time.

15. The method of claim 11, wherein the solid chemistry is at least about 99 wt-% dissolved in the pre-determined volume of water.

16. A method of cleaning and/or sanitizing a surface comprising:
obtaining the concentrate composition from the method of claim 1; and
contacting a hard surface in need of cleaning and/or sanitizing.

17. The method of claim 16, further comprising diluting the concentrate composition into a use solution before contacting a surface in need of cleaning, wherein the concentrate composition is diluted into the use solution at a ratio of between about 1:10 and about 1:10,000 concentrate to water.

18. The method of claim 16, wherein the hard surface in need of cleaning comprises a food processing surface.

19. The method of claim 16, wherein the hard surface is a clean-in-place system.

20. The method of claim 16, wherein the hard surface is part of a dairy operation or facility.
